(19) United States
(12) Patent Application Publication
(10) Pub. No.: US 2018/0360703 A1
(43) Pub. Date: Dec. 20, 2018

(54) COMPOSITION FOR COATING KERATIN FIBERS WITH AT LEAST ONE ESTER OF A FATTY ACID AND POLYOL

(71) Applicant: L'OREAL, Paris (FR)

(72) Inventors: Claudia BARBA, Paris (FR); Carole LEMERRER, Chevilly Larue (FR)

(21) Appl. No.: 16/061,095

(22) PCT Filed: Dec. 16, 2016
(86) PCT No.: PCT/EP2016/081421

§ 371 Date: Jun. 11, 2018

(30) Foreign Application Priority Data
Dec. 16, 2015 (FR) 15 62524

(51) Int. Cl. A61K 8/06; A61Q 1/10; A61K 8/37; A61K 8/92; A61K 8/81

(52) U.S. Cl. CPC A61K 8/062; A61Q 1/10; A61K 2800/43; A61K 8/922; A61K 8/8111; A61K 8/375

(57) ABSTRACT

The present invention relates to a composition, particularly for coating keratin fibers, notably eyelashes, in the form of a wax-in-water emulsion, comprising at least one pasty fat chosen from (poly)esters of at least one C₂₀-C₁₀₀ fatty acid, preferably behenic acid, and at least one polyol, preferably glycerol, wherein the wax content is strictly greater than 5% by weight with respect to the total weight of said composition.

The present invention relates to a cosmetic composition for coating keratin fibers, and in particular eyelashes or eyebrows, comprising at least one ester of a fatty acid and polyol. In particular, said cosmetic composition is a makeup composition, and possibly eyelash care composition. This invention also relates to a method for coating keratin fibers, in particular a makeup method, and possibly eyelash care method.

The composition implemented can in particular have the form of a product for eyelashes such as a mascara, or of a product for eyebrows. More preferentially, the invention concerns a mascara. The term “mascara” denotes a composition intended to be applied on the eyelashes. This can be a makeup composition for the eyelashes, a makeup base for eyelashes (also called a base-coat), a composition to be applied on a mascara (also called a top-coat), or a composition for the cosmetic treatment of eyelashes. The mascara is more particularly intended for the eyelashes of human beings, but also for false eyelashes.

Mascaras are particularly prepared according to two types of formulation:
aqueous mascaras referred to as “cream mascaras”, in the form of dispersion of waxes in water; and
anhydrous or low water content mascaras, referred to as “waterproof mascaras”, in the form of dispersions of waxes in organic solvents

The present application relates more specifically to aqueous mascaras. Compositions for coating keratin fibers such as aqueous mascara typically consist of at least one oily phase generally formed from one or a plurality of waxes dispersed in an aqueous liquid phase by means of an emulsifying system, or carried in an organic solvent.

At the present time, there is a need for a novel cosmetic composition for coating keratin fibers suitable for obtaining a deposit with satisfactory color properties.

One aim of the present invention is thus that of obtaining a composition for coating keratin fibers, preferably a mascara, having a satisfactory black intensity, in colorimetric and chromatic terms.

A further aim of the present invention is that of obtaining a composition for coating keratin fibers, preferably a mascara, providing a thickening of the eyelashes and a smooth deposit.

As such, the present invention relates to a composition, particularly for coating keratin fibers, notably eyelashes, in the form of a wax-in-water emulsion, comprising at least one pasty fat (C1) chosen from (poly)esters of at least one C₂₀-C₁₀₀ fatty acid, preferably behenic acid, and at least one polyol, preferably glycerol,

wherein the wax content is strictly greater than 5% by weight with respect to the total weight of said composition.

The composition according to the invention is in the form of a wax-in-water emulsion and is typically a mascara composition, more particularly an aqueous is mascara composition.

The composition according to the invention as defined above is suitable for obtaining a deposit on coated eyelashes with a satisfactory black intensity, notably enhanced with respect to a mascara composition without the specific pasty fat according to the invention.

Oily Phase

The composition according to the invention comprises an oily phase. In particular, the composition according to the invention, and more particularly the oily phase of a composition according to the invention, comprises at least one pasty fat and at least one wax.

Pasty Fats

A composition according to the invention comprises at least one pasty fat.

For the purposes of the invention, the term “pasty fat” refers to a lipophilic fat compound having a reversible solid/liquid change of state and comprising at a temperature of 23° C., a liquid fraction and a solid fraction.

In other words, the initial melting point of the pasty compound may be less than 23° C. The liquid fraction of the pasty compound measured at 23° C. may represent 9% to 97% by weight of the compound. This liquid fraction at 23° C. preferably represents between 15% and 85%, more preferably between 40% and 85% by weight.

Preferably, the pasty fat(s) has/have an ending melting point less than 60° C.

Preferably, the pasty fat(s) has/have a hardness less than or equal to 6 MPa.

Preferably, the pasty fat(s) has/have, in the solid state, a crystalline organization, visible by means of X-ray diffraction characterization.

According to the invention, the melting point is equivalent to the temperature of the most endothermic peak observed in thermal analysis (DSC) as described in the standard ISO 11357-3; 1999. The melting point of a paste or a wax may be measured using a differential scanning calorimeter (DSC), for example the calorimeter sold under the name “DSC 02000” by TA Instruments.

In respect of the melting point measurement and the determination of the ending melting point, the sample preparation and measurement protocols are as follows:

A 5 mg sample of pasty fat previously heated to 80° C. and sampled under magnetic stirring using a spatula which has also been heated is placed in a sealed aluminum capsule or crucible. Two tests are conducted to ensure the reproducibility of the results.

The measurements are made on the above-mentioned calorimeter. The oven is subject to nitrogen scavenging. Cooling is carried out by the RCS 90 heat exchanger. The sample is then subjected to the following protocol after stabilizing at a temperature of 20° C., and subjected to a first temperature rise from 20° C. to 80° C., at a heating rate of 5° C./minute, and is then cooled from 80° C. to −80° C. at a cooling rate of 5° C./minute and finally subjected to a second temperature rise from −80° C. to 80° C. at a heating rate of 5° C./minute. During the second temperature rise, the variation in the difference in power absorbed by the empty crucible and by the crucible containing the paste or wax sample as a function of temperature is measured. The melting point of the compound is the value of the temperature equivalent to the top point of the peak of the curve representing the variation in the difference in power absorbed as a function of temperature.

The ending melting point is equivalent to the temperature at which 95% of the sample has melted.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is said to be in the solid state when the entire mass thereof is in solid crystalline form. The pasty compound is said to be in the liquid state when the entire mass thereof is in liquid form.

The enthalpy of fusion of the pasty compound is equal to the integral of the overall fusion curve obtained using the above-mentioned calorimeter, with a temperature rise of 5° C. or 10° C. per minute, according to the ISO 11357-3:1999 standard. The enthalpy of fusion of the pasty compound is the quantity of energy required to change the compound from the solid state to the liquid state. It is expressed in J/g.

The enthalpy of fusion consumed at 23° C. is the quantity of energy required by the sample to change from the solid state to the state presented at 23° C. consisting of a liquid fraction and a solid fraction.

The liquid fraction of the pasty compound measured at 32° C. preferably represents 30% to 100% by weight of the compound, preferably 50% to 100%, more preferably 60% to 100% by weight of the compound. If the liquid fraction of the pasty compound measured at 32° C. is equal to 100%, the temperature of the end of the melting range of the pasty compound is less than or equal to 32° C.

The liquid fraction of the pasty compound at 32° C. is equal to the ratio of the enthalpy of fusion consumed at 32° C. to the enthalpy of fusion of the pasty compound. The enthalpy of fusion consumed at 32° C. is calculated as for the enthalpy of fusion consumed at 23° C.

In respect of the hardness measurement, the sample preparation and measurement protocols are as follows:

The pasty fat is placed in a 75 mm diameter mold filled to approximately 75% of the height thereof. So as not to be subject to the thermal history and to control crystallization, the mold is placed in a Votsch VC0018 programmable chamber where it is first set to a temperature of 80° C. for 60 minutes, and then cooled from 80° C. to 0° C. at a cooling rate of 5° C./minute, and then left at the stabilized temperature of 0° C. for 60 minutes, and subjected to a temperature rise from 0° C. to 20° C., at a heating rate of 5° C./minute, and then left at the stabilized temperature of 20° C. for 180 minutes.

The compression force measurement is made with the Swantech TA/TX2i texture analyzer. The spindle used is chosen according to the texture:
2 mm diameter cylindrical steel moving body for very rigid raw materials;
12 mm diameter cylindrical steel spindle for non-rigid raw materials;

The measurement comprises 3 steps: a first step after automatic detection of the surface of the sample on which the spindle moves at a measurement speed of 0.1 mm/s, and enters the pasty fat at a penetration depth of 0.3 mm, the software records the maximum force value reached; a second “relaxation” step wherein the spindle remains in this position for one second and wherein the force is recorded after 1 second of relaxation; finally, a 3^{rd} “withdrawal” step wherein the spindle returns to the initial position thereof at a speed of 1 mm/s and the probe withdrawal energy (negative force) is recorded.

The hardness value measured in the first step is equivalent to the maximum compression force measured in Newton divided by the surface area of the texture analyzer cylinder expressed in mm² in contact with the pasty fat. The hardness value obtained is expressed in megaPascal or MPa.

A composition according to the invention particularly comprises at least one is pasty fat (C1) chosen from (poly)esters of a fatty acid and polyol.

(Poly)Ester of Fatty Acid and Polyol

The composition according to the invention comprises at least one (poly)ester of at least one fatty acid comprising 20 to 100 carbon atoms, and at least one polyol.

According to the invention, the (poly)ester of fatty acid and polyol may be a mono-, di- or a tri-ester, or a mixture thereof.

Preferably, the fatty acid is chosen from the fatty acids wherein the linear or branched carbon chain comprises 20 to 50, preferably 20 to 30, and preferentially 20 to 25, carbon atoms.

According to one advantageous embodiment, the fatty acid according to the invention is chosen from C22 fatty acids, and is preferentially behenic acid.

According to the invention, the polyol may be notably chosen from diols. The diol may be chosen from aliphatic, alicyclic or aromatic diols. Use will preferably be made of a diol chosen from: ethylene glycol, diethylene glycol, triethylene glycol, 1,3-propanediol, cyclohexane dimethanol, 4-butanediol. As other polyols, glycerol, pentaerythritol, sorbitol, trimethylol propane may be used.

According to one advantageous embodiment, the polyol according to the invention is glycerol.

According to one embodiment, the composition according to the invention comprises at least one pasty fat (C1) chosen from (poly)esters of behenic acid and glycerol.

As such, the compound (C1) may be for example chosen from the mixtures of mono-, di- and/or tri-esters C₂₂ carboxylic acid(s) and glycerol.

The compound (C1) or ester of behenic acid and glycerol is notably chosen from the group consisting of glyceryl monobehenate, glyceryl di-behenate, glyceryl tri-behenate, and mixtures thereof. The carboxylic acid and glycerol ester may in particular be a mixture of glyceryl mono-behenate, glyceryl di-behenate and glyceryl tri-behenate.

Such a mixture is notably available, for example, under the INCI name “glyceryl dibehenate & tribehenin & glyceryl behenate”, and notably sold under the reference COMPRITOL 888 by GATTEFOSSE.

Preferably, the content of pasty fat (C1) in a composition according to the invention is greater than or equal to 0.1%, preferably ranging from 0.5% to 5%, and preferentially from 1% to 3%, by weight with respect to the total weight of said composition.

Additional Pasty Fat

The composition according to the invention may further comprise at least one additional pasty fat, i.e. a pasty fat different to C1.

Among the pasty fats, mention may be made for example of synthetic compounds and plant-based compounds. Such a pasty fat may be obtained by means of synthesis from plant-based starting materials.

This/these pasty fat(s) is/are advantageously chosen from:
lanolin and the derivatives thereof;
petroleum jelly, particularly that for which the INCI name is petrolatum and sold under the name ULTIMA WHITE PET USP by Perenco;
polyol ethers chosen from pentaerythritol and polyalkylene glycol ethers, fatty alcohol and sugar ethers, and mixtures thereof, pentaerythritol and polyethylene glycol ether comprising 5 oxyethylene units (5 OE) (CTFA name: PEG-5 Pentaerythrityl Ether), pentaerythritol and polypropylene glycol ether comprising 5 oxypropylene units (5 OP) (CTFA name: PPG-5 Pentaerythrityl Ether), and the mixtures thereof and more specifically the mixture of PEG-5 Pentaerythrityl Ether, PPG-5 Pentaerythrityl Ether and soybean oil, sold under the name “Lanolide” by Vevy, wherein the ratio of the constituents by weight is 46:46:8:46% PEG-5 Pentaerythrityl Ether, 46% PPG-5 Pentaerythrityl Ether and 8% soybean oil;
hydrogenated oils, notably hydrogenated palm oil and hydrogenated jojoba oil;
optionally polymeric silicone compounds;
optionally polymeric fluorinated compounds;
vinyl polymers, in particular:
   olefin homopolymers and copolymers;
   hydrogenated diene homopolymers and copolymers;
   linear or branched oligomers, alkyl (meth)acrylate homo or copolymers preferably having a C₈-C₃₀ alkyl group,
   vinyl ester homo and copolymer oligomers, having C₈-C₃₀ alkyl groups,
   vinyl ether homo and copolymer oligomers, having C₈-C₃₀ alkyl groups,
liposoluble polyethers derived from polyetherification between one or a plurality of C₂-C₁₀₀, preferably C₂-C₅₀, diols,
esters other than the compounds C1 mentioned above,
and/or mixtures thereof.

The composition according to the invention may comprise one or a plurality of additional pasty fat(s) with a total content greater than or equal to 0.01% by weight, with respect to the total weight of the composition, preferably between 0.1% and 5% by weight, preferentially from 0.1% to 2% by weight, with respect to the total weight of the composition.

According to one embodiment, a composition according to the invention comprises at least one additional pasty fat chosen from hydrogenated oils, notably hydrogenated palm oil and hydrogenated jojoba oil, glycerol oligomer esters, and mixture(s) thereof.

According to one particular embodiment, a composition according to the invention comprises at least one additional pasty fat chosen from glycerol oligomer esters, notably with a content greater than or equal to 0.1%, preferably between 0.5% and 5%, and preferentially between 1% and 3%, by weight with respect to the total weight of said composition.

The glycerol oligomer esters are notably chosen from diglycerol esters, particularly adipic acid and glycerol condensates, for which part of the hydroxyl groups of the glycerols have reacted with a mixture of fatty acids such as stearic acid, capric, stearic acid and isostearic acid and 12-hydroxystearic acid, preferably such as bis-diglyceryl polyacyladipate-2, for example sold under the brand Softisan 649 by Sasol.

Wax(es)

A composition according to the invention comprises at least one wax.

The wax or waxes considered in the framework of this invention is/are in general a lipophilic compound, which is solid at ambient temperature (25° C.), having a reversible solid/liquid change of state and a melting point greater than or equal to 30° C. of up to 200° C. and particularly up to 120° C.

In particular, the waxes suitable for the invention may have a melting point greater than or equal to 45° C., and particularly greater than or equal to 55° C.

According to the invention, the melting point is equivalent to the temperature of the most endothermic peak observed in thermal analysis (DSC) as described in the standard ISO 11357-3; 1999. The melting point of the wax may be measured using a differential scanning calorimeter (DSC), for example the calorimeter sold under the name “DSC 02000” by TA Instruments.

Preferably, the waxes have an enthalpy of fusion ΔHf greater than or equal to 70 J/g.

Preferably, the waxes comprise at least one part suitable for crystallization, visible by means of diffraction X-ray observations.

The measurement protocol is as follows:

A 5 mg sample of wax placed in a crucible is subjected to a first temperature rise from −20° C. to 120° C., at a heating rate of 10° C./minute, and is then cooled from 120° C. to −20° C. at a cooling rate of 10° C./minute and finally subjected to a second temperature rise from −20° C. to 120° C. at a heating rate of 5° C./minute. During the second temperature rise, the following parameters are measured:
the melting point (T_{f}) of the wax, as mentioned above equivalent to the temperature of the most endothermic peak of the fusion curve observed, representing the variation in the difference in power absorbed as a function of the temperature,
ΔHf: the enthalpy of fusion of the wax equivalent to the integral of the overall fusion curve obtained. This enthalpy of fusion of the wax is the quantity of energy required to change the compound from the solid state to the liquid state. It is expressed in J/g.

The wax or waxes can be hydrocarbon, fluorinated and/or silicone and be of plant, mineral, animal, and/or synthetic origin.

As wax(es), preference can be given to the use of hydrocarbon waxes such as beeswax, lanolin wax, and Chinese insect waxes, rice bran wax, Carnauba wax, Candellila wax, Ouricury wax, Alfa wax, cork fiber wax, sugarcane wax, Japan wax and sumac wax; montan wax, microcrystalline waxes, paraffins and ozokerite waxes; polyethylene waxes, waxes obtained by means of Fisher-Tropsch synthesis and waxy copolymers and the esters thereof.

Mention may also be made of waxes obtained by means of the catalytic is hydrogenation of animal or plant oils having C₈-C₃₂ linear or branched fat chains.

Of these, particular mention may be made of isomerized jojoba oil such as the trans isomerized partially hydrogenated jojoba oil manufactured or sold by Desert Whale under the trade name ISO-JOJOBA-50®, hydrogenated sunflower oil, hydrogenated castor oil, hydrogenated coconut oil, hydrogenated lanolin oil, di-(trimethylol-1,1,1 propane) tetrastearate sold under the name “HEST 2T-4S” by HETERENE, di-(trimethylol-1,1,1 propane) tetrabehenate sold under the name HEST 2T-4B by HETERENE.

Silicon waxes can also be mentioned such as alkyl dimethicones or alkoxy dimethicones having 16 to 45 carbon atoms, fluorinated waxes.

The wax can also be used obtained by hydrogenating esterified olive oil with stearyl alcohol sold under the name “PHYTOWAX Olive 18 L 57” or waxes obtained by hydrogenating esterified castor oil with cetyl alcohol sold under the name “PHYTOWAX ricin 16L64 and 22L73”, by SOPHIM. Such waxes are described in the application FR-A-2792190.

According to one embodiment, the wax(es) are chosen from one or a plurality of polar and/or non-polar waxes.

The composition can comprise at least one non-polar wax. Preferably, the wax or waxes comprise(s) one or a plurality of non-polar waxes chosen from among polyethylene wax, paraffin wax, ozokerite, and mixtures thereof.

The composition can comprise at least one polar wax. The term “polar wax” denotes waxes that comprise in their chemical structure, in addition to carbon and hydrogen atoms, at least one highly electronegative heteroatom, such as 0, N or P.

According to one embodiment, a composition according to the invention comprises a mixture of polar and non-polar wax(es), preferably chosen from among carnauba wax, candellila wax, natural (or bleached) beeswax and synthetic beeswax, paraffin and hydrocarbon wax, and mixtures thereof.

As synthetic beeswax, mention can be made of the wax sold under the name Cyclochem 326 A by Evonik Goldschmidt (INCI name: Synthetic Beeswax).

According to one embodiment, the wax content in a composition according to the invention is strictly greater than 5% by weight with respect to the total weight of the composition, particularly ranging from 5.5% to 20% by weight, preferably from 6% to 15% by weight, and preferentially from 7% to 10%, by weight with respect to the total weight of said composition.

Lipophilic Gelling Agent

The composition may further comprise at least one lipophilic gelling agent.

Preferably, this lipophilic gelling agent is chosen from lipophilic polyamide polycondensates.

The term “polycondensate” refers in terms of the invention to a polymer obtained through polycondensation namely by chemical reaction between monomers that have different functional groups chosen in particular from among the acid, alcohol and amine functions.

The term “polymer” in terms of the invention means a compound that has at least 2 repeat units, preferably at least 3 repeat units and more preferably 10 repeat units.

The lipophilic polyamide polycondensates may in particular be chosen from among the polyamide polymers comprising a) polymeric backbone that has hydrocarbon repeat units provided with at least one non-pendant amide pattern, and possibly b) at least one pendant fatty chain comprising from 6 to 120 carbon atoms, preferably from 8 to 120 carbon atoms, and more preferably from 12 to 70 carbon atoms, and/or at least one terminal fatty chain that may be functionalized, comprising at least 4 carbon atoms and being linked to these hydrocarbon patterns.

The term “functionalized chains” in terms of the invention refers to an alkyl chain comprising one or several functional group or reagents in particular chosen from among the amides, hydroxyl, ether, oxyalkylene or polyoxyalkylene groups, halogen, of which the fluorinated or perfluorinated groups, ester, siloxane, polysiloxane. Furthermore, the hydrogen atoms of one or several fatty chains can be substituted at least partially with fluorine atoms.

The term “hydrocarbon repeat units” in terms of the invention refers to a unit comprising from 2 to 80 carbon atoms, and preferably from 2 to 60 carbon atoms, carrying hydrogen atoms and possibly oxygen atoms, which can be linear, branched or cyclic, saturated or unsaturated. These units further comprise at least one amide group advantageously non-pendant and found in the polymeric backbone.

Advantageously, the pendant chains are directly linked to at least one of the nitrogen atoms of the polymeric backbone.

The lipophilic polyamide polycondensate can comprise between the is hydrocarbon patterns silicone units or oxyalkylene units in C₂-C₃.

Furthermore, the lipophilic polyamide polycondensate of the composition of the invention advantageously comprises from 40 to 98% of the fatty chains with respect to the total number of amide units and fatty chains and more preferably from 50 to 95%.

Preferably, the pendant fatty chains are linked to at least one of the atoms of nitrogen of the amide units of the polymer. In particular, the fatty chains of this polyamide represent from 40 to 98% of the total number of amide units and of the fatty chains, and more preferably from 50 to 95%.

Advantageously, the lipophilic polyamide polycondensate has a mean molecular weight by weight less than 100,000 (in particular ranging from 1000 à 100,000), in particular less than 50,000 (in particular ranging from 1000 to 50,000), and more particularly ranging from 1000 to 30,000, preferably from 2000 to 20,000, and more preferably from 2000 to 10,000.

The lipophilic polyamide polycondensate is not soluble in water, in particular at 25° C. In particular, it does not comprise an ionic group.

As preferred lipophilic polyamide polycondensates that can be used in the invention, mention can be made of polyamides branched by pendant fatty chains and/or terminal fatty chains having from 6 to 120 carbon atoms and more particularly from 8 to 120 and in particular from 12 to 68 carbon atoms, with each terminal fatty chain being linked to the polyamide backbone by at least one bond group L. The bond group L can be chosen from among the ester, ether, amine, urea, urethane, thioester, thioether, thiourea, thiourethane groups. Preferably, these polymers comprise a fatty chain at each end of the polyamide backbone.

These polymers are preferably polymers resulting from a polycondensation between a carboxylic diacid having at least 32 carbon atoms (that has in particular from 32 to 44 carbon atoms) with an amine chosen from among diamines that have at least 2 carbon atoms (in particular from 2 to 36 carbon atoms) and triamines having at least 2 carbon atoms (in particular from 2 to 36 carbon atoms). The diacid is preferably a dimer coming from ethylene unsaturation fatty acid having at least 16 carbon atoms, preferably from 16 to 24 carbon atoms, such as oleic, linoleic or linolenic acid. The diamine is preferably diamine ethylene, diamine hexylene, diamine hexamethylene. The triamine is for example triamine ethylene. For polymers comprising one or 2 terminal carboxylic acid groups, it is advantageous to esterify them with a monoalcohol that has at least 4 carbon atoms, preferably from 10 to 36 carbon atoms and more preferably from 12 to 24 and even more preferably from 16 to 24, for example 18 carbon atoms.

The lipophilic polyamide polycondensate of the composition according to the invention may particularly be chosen from among the polymers having the following formula (A):

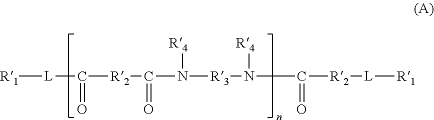

in which:
n is an integer ranging from 1 to 30,
R′₁ represents at each occurrence independently a fatty chain and is chosen from among an alkyl or alkenyl group having at least 4 carbon atoms and in particular from 4 to 24 carbon atoms;
R′₂ represents at each occurrence independently a divalent hydrocarbon chain comprising from 1 to 52 carbon atoms;
R′₃ represents at each occurrence independently a divalent hydrocarbon group, saturated or unsaturated, cyclic or acyclic, possibly substituted and/or possibly interrupted by one or several heteroatoms preferably chosen from among oxygen and nitrogen comprising at least one carbon atom, in particular R′₃ represents a linear or branched alkylene chain (C₁-C₈); preferably linear alkylene (C₁-C₆) such as ethylene;
R′₄ represents at each occurrence independently: a hydrogen atom, one alkyl group comprising from 1 to 10 carbon atoms, or a direct link with at least one group chosen from among R′₃ and another R′₄ in such a way that when said group is another R′₄, the nitrogen atom to which are linked both R′₃ and R′₄ is part of a heterocyclic structure defined by R′₄—N—R′₃, with the condition that at least 50% of the R′₄ represent a hydrogen atom, and
L represents a bond group chosen preferably from among an ester or ether or amine or urea or urethane or thioester or thioether or thiurea or thiourethane group, possibly substituted by at least one group R′₁ as defined hereinabove.

According to one embodiment, these polymers are chosen from among the polymers having formula (A) wherein the bond group L represents a —C(O)—O— or —O—C(O)— ester group.

These polymers are more specifically those described in the document U.S. Pat. No. 5,783,657 from Union Camp.

Each one of these polymers satisfies in particular the following formula (B):

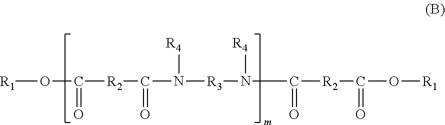

in which:
m designates an integer of an amide group such that the number of the ester groups represents from 10% to 50% of the total number of ester and amide groups;
R₁ represents at each occurrence independently an alkyl or alkenyl group having at least 4 carbon atoms and in particular from 4 to 24 carbon atoms;
R₂ represents at each occurrence independently a C₄ to C₄₂ hydrocarbon group with the condition that 50% of the groups R₂ represent a C₃₀ to C₄₂ hydrocarbon group;
R₃ represents at each occurrence independently a divalent hydrocarbon group, saturated or unsaturated, cyclic or acyclic, possibly substituted and/or possibly interrupted by one or several heteroatoms preferably chosen from among oxygen and nitrogen comprising at least one carbon atom, in particular R₃ represents a linear or branched alkylene chain (C₁-C₈); preferably linear alkylene (C₁-C₆) such as ethylene;
and R₄ represents at each occurrence independently a hydrogen atom, a C₁ to C₁₀ alkyl group or a direct link to R₃ or to another R₄ in such a way that the nitrogen atom to which are linked both R₃ and R₄ is a part of a heterocyclic structure defined by R₄—N—R₃, with at least 50% of the R₄ representing a hydrogen atom.

In the particular case of the formula (B), the terminal fatty chains possibly functionalized in terms of the invention are terminal chains linked to the last nitrogen atom, of the polyamide backbone.

In particular, the ester groups of the formula (B), which are part of the terminal and/or pendant fatty chains in terms of the invention, represent from 15% to 40% of the total number of ester and amide groups and preferably from 20% to 35%.

In addition, m advantageously represents an integer ranging from 1 to 5 and more particular greater than 2.

Preferably, R₁ is a C₁₂ to C₂₂ alkyl group and preferably C₁₆ to C₂₂. Advantageously, R₂ can be a divalent, saturated or unsaturated, cyclic or acyclic hydrocarbon group in particular R₂ represents a linear or branched (C₁₀-C₄₂) alkylene chain. Preferably, 50% at least and more preferably at least 75% of the radicals R₂ are groups having 30 to 42 carbon atoms. The other R₂ are hydrocarbon groups in C₄ to C₁₉ and even in C₄ to C₁₂.

Preferably, R₃ represents a C₂ to C₃₆ hydrocarbon group or polyoxyalkylene group and R₄ represents a hydrogen atom. Preferably, R₃ represents a C₂ to C₁₂ hydrocarbon group.

The hydrocarbon groups can be linear, cyclic or branched, saturated or unsaturated groups. Moreover, the alkyl and alkylene groups can be linear or branched, saturated or not, groups.

In general, the polymers of the formula (B) have the form of mixtures of polymers, with these mixtures furthermore able to contain a synthesis product that corresponds to a compound of formula (B) where n is equal to 0, i.e. a diester.

According to a particularly preferred form of the invention, a mixture of copolymers of a C36 diacid condensed on diamine ethylene will be used; the terminal ester groups result from the esterification of the terminations of remaining acid by cetyl, stearyl alcohol or mixtures thereof (also called cetostearyl alcohol) (INCI name: ETHYLENEDIAMINE/STEARYL DIMER DILINOLEATE COPOLYMER). Its mean molar mass by weight is preferably 6,000, more preferentially 4,000. These mixtures are in particular sold by ARIZONA CHEMICAL under the trade names UNICLEAR 80 and UNICLEAR 100 VG respectively in the form of a gel at 80% (in active material) in a mineral oil and at 100% (in active material). These mixtures are also sold by CRODA under the trade name OLEOCRAFT LP-10-PA-(MV) respectively at 99.7% (in active material) with a preservative. They have a softening point from 88° C. to 94° C.

In terms of polyamide polycondensates that satisfy the general formula (A), mention can also be made of polymers comprising at least one terminal fatty chain linked to the polymeric backbone by at least one tertiary amide bond group (also called amide terminated polyamide or ATPA). For more information on these polymers, reference can be made to document U.S. Pat. No. 6,503,522.

According to a particularly preferred form of the invention, use will be made more particularly of a copolymer of hydrogenated linoleic diacid, ethylenediamine, di(C14-C18)alkylamine(s) (INCI name: ETHYLENEDIAMIDE/HYDROGENATED DIMER DILINOLEATE COPOLYMER BIS-DI-C14-C18 ALKYL AMIDE). This copolymer is in particular sold under the trade name SYLVACLEAR A200V by ARIZONA CHEMICAL.

According to another embodiment, the polyamide having formula (A) can also be a poly(ester-amide) with ester ends (ester-terminated poly(ester-amide) or ETPEA), as for example those for which the preparation is described in document U.S. Pat. No. 6,552,160.

According to a particularly preferred form of the invention, use will be made more particularly of a copolymer of hydrogenated linoleic diacid, ethylenediamine, neopentylglycol and stearyl alcohol (INCI name: BIS-STEARYL ETHYLENEDIAMINE/NEOPENTYL GLYCOL/STEARYL HYDROGENATED DIMER DILINOLEATE COPOLYMER). This copolymer is in particular sold under the trade name SYLVACLEAR C75 V by ARIZONA CHEMICAL.

As polyamide polycondensates that can be used in the invention, further mention can be made of those that contain at least one terminal fatty chain linked to the polymeric backbone by at least one ether or polyether bond group (it is then referred to as ether terminated poly(ether)amide). Such polymers are described for example in the document U.S. Pat. No. 6,399,713.

The polyamides in accordance with the invention advantageously have a softening temperature greater than 65° C. and are able to range up to 190° C. Preferably, it has a softening temperature ranging from 70° C. to 130° C. and more preferably from 80° C. to 105° C. The polyamide is in particular a non-waxy polymer.

As polyamide polycondensates that can be used in the invention, mention can also be made of polyamide resins resulting from the condensing of a aliphatic di-carboxylic acid and of a diamine (including the compounds that have more than 2 carbonyl groups and 2 amine groups), with the carbonyl and amine groups of adjacent unitary units being condensed by an amide bond. These polyamide resins are in particular those sold under the brand Versamid by General Mills, Inc. and Henkel Corp. (Versamid 930, 744 or 1655) or by Olin Mathieson Chemical Corp., under the brand Onamid in particular Onamid S or C. These resins have a mean molecular mass by weight ranging from 6,000 to 9,000. For more information on these polyamides, reference can be made to documents U.S. Pat. No. 3,645,705 and U.S. Pat. No. 3,148,125. More specifically, Versamid 930 or 744 are used.

Polyamides sold by Arizona Chemical under references Uni-Rez (2658, 2931, 2970, 2621, 2613, 2624, 2665, 1554, 2623, 2662) can also be used and the product sold under the reference Macromelt 6212 by Henkel. For more information on these polyamides, reference can be made to document U.S. Pat. No. 5,500,209.

It is also possible to use resins of polyamides coming from vegetables such as those described in patents U.S. Pat. No. 5,783,657 and U.S. Pat. No. 5,998,570.

Preferably, this lipophilic gelling agent is a copolymer of a C36 diacid condensed on diamine ethylene; the terminal ester groups result from the esterification of the terminations of remaining acid by cetyl, stearyl alcohol or mixtures thereof.

The lipophilic gelling agent polymer(s) may be present in the composition in a quantity of active material ranging from 0.05% to 12% by weight, preferably from 0.1% to 5% by weight relative to the total weight of the composition.

Polybutene

The composition according to the invention may further comprise polybutene.

The polybutene optionally used in a composition according to the invention is preferably equivalent to a non-volatile oil.

The term “oil” denotes a liquid fat at an ambient temperature of 25° C. and at atmospheric pressure.

The term “non-volatile oil” denotes an oil remaining on the skin or keratin fiber at ambient temperature and pressure. More precisely, a non-volatile oil has an evaporation ratestrictlybelow 0.01 mg/cm²/min.

To measure this evaporation rate, 15 g of oil or an oil mixture to be tested are introduced into a crystallizer with a diameter of 7 cm, placed on a scale located in a large chamber of around 0.3 m³, with controlled temperature, at 25° C., and hygrometry, at 50% relative humidity. The liquid is left to evaporate freely, without stirring, by allowing ventilation with a fan (PAPST-MOTOREN, reference 8550 N, rotating at 2700 rpm) arranged vertically above the crystallizer containing said oil or said mixture, with the blades being directed toward the crystallizer and at a distance of 20 cm with respect to the crystallizer base. The mass of oil remaining in the crystallizer is measured at regular intervals. The evaporation rates are expressed in mg of oil evaporated per unit of surface (cm²) and per unit of time (minutes).

A polybutene according to the invention advantageously has at least 10 monomers, preferentially between 12 and 50 monomers, more preferentially between 15 and 40 monomers.

A polybutene according to the invention has a molecular mass by weight Mw greater than or equal to 750 g/mol, advantageously between 800 and 10,000 g/mol, more preferentially between 900 and 5,000 g/mol.

Preferably, said polybutene comprises at least one monomer chosen from the 4 structural isomers of butene, i.e. but-1-ene, (Z)-but-2-ene, (E)-but-2-ene, 2-methylprop-1-ene (or isobutene), and mixtures thereof. The term polybutene covers polymers derived from at least any one of these monomers hereinafter in this description.

Preferably, said polybutene comprises at least one monomer of 2-methylprop-1-ene.

According to one particular embodiment, said polybutene is derived from the copolymerization of at least two of these 4 isomers. Preferably, one of these two isomers is 2-methylprop-1-ene. According to one particular embodiment, said resulting polybutene comprises a mixture of monomers of polybut-1-ene and 2-methylprop-1-ene.

Preferably, said polybutene is partially hydrogenated.

The total polybutene content according to the invention ranges from 0.1% to 10% by weight, particularly from 0.5% to 5% by weight, and preferably from 1% to 3% by weight, with respect to the total weight of the composition.

The polybutene according to the invention advantageously exhibits at 100° C. a kinematic viscosity greater than 1,000 centistokes, preferably between 2,000 and 8,000 centistokes, more preferentially between 2,500 and 5,000 centistokes.

The polybutene according to the invention exhibits at 25° C. a static viscosity greater than or equal to 20,000 mPa·s, preferably greater than or equal to 65,000 mPa·s, preferably greater than or equal to 350,000 mPa·s, for example inclusively between 25,000 and 800,000 mPa·s, preferably between 400,000 and 600,000 mPa·s. Such a viscosity can be measured using an RS75 rheometer sold by HAAKE equipped with a plate or cone 60 mm in diameter inclined by 2°.

The polybutene used within the scope of the present invention may for example be chosen from Indopol H-100, Indopol H-300, Indopol H-1500 from Amoco, Parleam V, Parleam HV and Parleam SV from NOF Corporation. Preferentially, Indopol H-1500 or Parleam SV will be used.

Aqueous Phase

A composition according to the invention comprises an aqueous phase, preferably forming a continuous phase of the composition.

The aqueous phase comprises water. It may also comprise at least one water-soluble solvent.

The term “water-soluble solvent” denotes in the present invention a water-miscible liquid compound at ambient temperature.

The water-soluble solvents suitable for use in the compositions according to the invention may further be volatile.

Of the water-soluble solvents suitable for use in the compositions according to the invention, mention may notably be made of lower monoalcohols having 1 to 5 carbon atoms such as ethanol and isopropanol; glycols having 2 to 8 carbon atoms such as ethylene glycol, propylene glycol, 1,3-butylene glycol and dipropylene glycol.

The aqueous phase (water and optionally the water-miscible solvent) is generally present in the composition according to the present application with a content ranging from 20% to 90% by weight, with respect to the total weight of the composition, preferably ranging from 25% to 80% by weight, preferentially ranging from 30% to 70% by weight, and more preferentially from 35% to 60% by weight with respect to the total weight of the composition.

According to one advantageous embodiment, in a composition according to the invention, the water content is greater than or equal to 40%, or greater than or equal to 45%, by weight with respect to the total weight of said composition.

Film-Forming Polymer

The composition according to the invention may further comprise at least one film-forming polymer.

The compositions according to the invention may particularly comprise at least one water-soluble film-forming polymer, at least one film-forming polymer in the form of particles in aqueous dispersion, and the mixture thereof, more particularly a mixture of at least one water-soluble film-forming polymer and at least one film-forming polymer in the form of particles in aqueous dispersion.

According to one embodiment, a composition according to the invention comprises a total film-forming polymer content greater than or equal to 0.1%, particularly ranging from 1% to 15%, more particularly from 2% to 8%, by weight with respect to the total weight of the composition.

In this application, the term “film-forming polymer” denotes a polymer suitable for forming alone or in the presence of an auxiliary film-forming agent, a macroscopically continuous deposit, and preferably a cohesive deposit, and more preferably a deposit for which the cohesion and mechanical properties are such that said deposit can be isolated and manipulated in an isolated manner, for example when said deposit is embodied by pouring on a non-stick surface such as a Teflon or silicone surface.

The film-forming polymer(s) may be in the form of dispersion in an aqueous medium.

The film-forming polymer(s) present in said preparation of the composition in the form of particles in aqueous dispersion, generally bear(s) the name of (pseudo)latex, i.e. latex or pseudolatex. The techniques for preparing these dispersions are well known to those skilled in the art. A dispersion suitable for the invention may comprise one or a plurality of types of particles, these particles optionally varying by the size thereof, by the structure thereof and/or by the chemical nature thereof.

The term “aqueous dispersion” denotes a liquid medium based on water and/or hydrophilic solvents. This aqueous liquid medium may consist essentially of water. It may also comprise a mixture of water and water-miscible organic solvent(s) (water-miscibility greater than 50% by weight at 25° C.).

Among the film-forming polymers that can be used in the composition of this invention, mention may be made of radical or polycondensate type synthetic polymers, polymers of natural origin, and mixtures thereof.

By way of examples of water-soluble film-forming polymers, mention may be made of:
proteins like proteins of plant origin such as wheat, soy proteins; proteins of animal origin such as keratins, for example keratin hydrolysates and sulfonic keratins;
cellulose polymers such as hydroxyethylcellulose, hydroxypropylcellulose, methylcellulose, ethylhydroxyethylcellulose, carboxymethylcellulose, and quaternized cellulose derivatives;
acrylic polymers or copolymers, such as polyacrylates or polymethacrylates;
vinyl polymers, such as polyvinylpyrrolidones, methylvinyl ether and malic anhydride copolymers, vinyl acetate and crotonic acid copolymer, vinylpyrrolidone and vinyl acetate copolymers; vinylpyrrolidone and caprolactam copolymers; polyvinyl alcohol;
anionic, cationic, amphoteric or non-ionic chitin or chitosan polymers;
gum arabic, guar gum, xanthan derivatives, karaya gum;
alginates and carrageenans;
glycosaminoglycans, hyaluronic acid and derivatives thereof;
shellac resin, sandarac gum, dammars, elemis, copals;
deoxyribonucleic acid;
mucopolysaccharides such as chondroitin sulfates;
and mixtures thereof.

According to one embodiment, a composition according to the invention comprises a total water-soluble film-forming polymer content greater than or equal to 0.1% by weight, particularly ranging from 0.5% to 5%, by weight with respect to the total weight of the composition.

By way of aqueous film-forming polymer dispersion, use may be made of acrylic dispersions sold under the names Neocryl XK-90®, Neocryl A-1070®, Neocryl A-1090®, Neocryl BT-62®, Neocryl A-1079® and Neocryl A-523® by AVECIA-NEORESINS, Dow Latex 432® by DOW CHEMICAL, Daitosol 5000 AD® or Daitosol 5000 SJ® by DAITO KASEY KOGYO; Syntran 5760® by Interpolymer Allianz Opt® by Rohm and Haas; acrylic acid/ethylene copolymer dispersions notably sold under the name Asensa® SC 401 by HONEYWELL; aqueous polyurethane dispersions sold under the names Neorez R-981® and Neorez R-974® by AVECIA-NEORESINS, Avalure UR-405®, Avalure UR-410®, Avalure UR-425®, Avalure UR-450®, Sancure 875®, Avalure UR-445® and Sancure 2060® by NOVEON, Impranil 85® by BAYER, Aquamere H-1511® by HYDROMER; sulfopolyesters sold under the brand name Eastman AQ® by Eastman Chemical Products, vinyl dispersions such as Mexomere PAM®, aqueous polyvinyl acetate dispersions such as “Vinybran®” from Nisshin Chemical or those sold by UNION CARBIDE, aqueous vinyl pyrrolidoneterpolymer dispersions, dimethylaminopropyl methacrylamide and lauryldimethylpropylmethacrylamidoammonium chloride such as Styleze W- from ISP, aqueous hybrid polyurethane/polyacrylic polymer dispersions such as those sold under the references “Hybridur®” by AIR PRODUCTS or “Duromer®” by NATIONAL STARCH, core/shell type dispersions: for example those sold by ATOFINA under the reference Kynar (core: fluorinated-shell: acrylic) or those described in the document U.S. Pat. No. 5,188,899 (core; silica-shell: silicone) and mixtures thereof.

According to one embodiment, a composition according to the invention comprises a total dry matter content of film-forming polymer particles in the form of aqueous dispersion greater than or equal to 0.1% by weight, in particular ranging from 1% to 10%, by weight with respect to the total weight of the composition.

Dyes

The composition according to the invention can comprise at least one dye.

This dye or these dyes is/are preferably chosen from the group constituted of powder materials, liposoluble dyes, water-soluble dyes, and mixtures thereof.

Preferably, the composition according to the invention comprises at least one powder dye. Preferably, the powder dye or dyes can be chosen from among pigments.

Preferably, the pigment or pigments contained in the composition according to the invention are chosen from among metal oxides, preferably iron oxides.

These dyes may be present with a content ranging from 0.01% to 20% by mass with respect to the total weight of the composition, in particular from 1% to 15% by mass, preferably from 3% by 10% by mass with respect to the total weight of the composition.

Preferably, the dye or dyes are chosen from among one or several metal oxides present with a content greater than or equal to 2% by weight with respect to the total weight of the composition, advantageously between inclusively 3% and 12% by weight with respect to the total weight of the composition.

Surfactant(s)

The composition according to the invention advantageously comprises an emulsifying system.

According to one embodiment, the emulsifying system comprises at least one surfactant or a mixture of surfactants.

These surfactants may be chosen from non-ionic surfactants, anionic surfactants, and mixtures thereof.

This emulsifying system may for example comprise, or consist of:
at least one non-ionic surfactant, particularly at least one non-ionic surfactant having an HLB less than 8 at 25° C.,
at least one anionic surfactant, and
optionally at least one co-surfactant.

This or these surfactant(s) are more generally present with a total content ranging from 4% to 14% by weight, and preferably from 6% to 12% by weight, with respect to the total weight of the composition.

An emulsifying surfactant suitable chosen for obtaining an oil-in-water emulsion is generally used.

The HLB value as per GRIFFIN is defined in J. Soc. Cosm. Chem. 1954 (volume 5), pages 249-256.

Reference may be made to the document “Encyclopedia of Chemical Technology, KIRK-OTHMER”, volume 22, p. 333-432, 3^{rd} edition, 1979, WILEY, for the definition of the properties and emulsifying functions of surfactants, in particular p. 347-377 of this reference, for anionic, amphoteric and non-ionic surfactants.

The surfactants used in the composition according to the invention are for example chosen from non-ionic surfactants having an HLB less than 8 at 25° C., for example chosen from:
sugar esters and ethers such as sucrose stearate, sucrose cocoate, sorbitan stearate and mixtures thereof, for example Arlatone 2121® sold by ICI or SPAN 65V from UNIQEMA;
esters of fatty acids, notably C₈-C₂₄, and preferably C₁₆-C₂₂, and polyol, notably of glycerol or sorbitol, such as glyceryl stearate, for example sold under the name TEGIN M® by GOLDSCHMIDT, glyceryl laurate such as the product sold under the name IMWITOR 312® by HULS, polyglyceryl-2 stearate, sorbitan tristearate, glyceryl ricinoleate;
oxyethylene and/or oxypropylene ethers such as the oxyethylene ether of stearyl alcohol with 2 oxyethylene units (CTFA name “Steareth-2”) such as BRIJ 72 sold by UNIQEMA; and
the cyclomethicone/dimethicone copolyol mixture sold under the name 02-32250® by DOW CORNING.

Preferably, the emulsifying system is chosen from non-ionic surfactants, preferably chosen from oxyethylene and/or oxypropylene ethers, and more preferentially comprises the oxyethylene ether of stearyl alcohol with 2 oxyethylene units (CTFA name “Steareth-2”).

The surfactants used in the composition according to the invention are for example chosen from anionic surfactants, for example chosen from alkylphosphates, and particularly C₁₂-C₂₄, preferably C₁₄-C₁₈ alkyl phosphate(s) and mixtures thereof, in particular they may be chosen from DEA oleth-10 phosphate (Crodafos N 10N from CRODA), cetyl phosphate (Amphisol K from Givaudan or ARLATONE MAP 160K from UNIQEMA), stearyl phosphate and cetearyl phosphate.

A composition according to the invention advantageously comprises one or a plurality of surfactants, for example chosen from fatty alcohols comprising from 10 to 26 carbon atoms, preferably from 12 to 24 carbon atoms and preferentially from 14 to 22 carbon atoms.

Preferably, the co-surfactant is cetyl alcohol.

The total surfactant content is preferably between 0.01% and 5% by weight with respect to the total weight of the composition.

Additives

The compositions in accordance with the invention can also comprise at least one additive.

By way of additives suitable for use in the compositions according to the invention, mention may in particular be made of antioxidants, preservatives, volatile oils, additional non-volatile oils, hydrophilic gelling agents, perfumes, neutralizers, emollients, coalescing agents, plasticizers, moisturizers, vitamins and mixtures thereof.

Obviously, those skilled in the art will take care to choose these optional additional compounds, and/or the quantity thereof, such that the advantageous properties of the active constituents of the composition according to the invention are not, or are substantially not, altered by the envisaged addition.

Cosmetic Compositions

The present invention also relates to a cosmetic composition including, in a physiologically acceptable medium, a composition as defined above.

The term “physiologically acceptable medium” is intended to denote a medium that is particularly suitable for the application of a composition of the invention to the skin, the eyelashes or the eyebrows.

The physiologically acceptable medium is generally suitable for the nature of the support to which the composition should be applied, and also for the way in which the composition is to be packaged.

The composition implemented can in particular have the form of a product for eyelashes such as a mascara, or of a product for eyebrows. More preferentially, the invention concerns a mascara. The term “mascara” denotes a composition intended to be applied on the eyelashes. This can be a makeup composition for the eyelashes, a makeup base for eyelashes (also called a base-coat), a composition to be applied on a mascara (also called a top-coat), or a composition for the cosmetic treatment of eyelashes. The mascara is more particularly intended for the eyelashes of human beings, but also for false eyelashes.

Applications

The present invention also relates to a non-therapeutic cosmetic method for coating keratin materials, in particular keratin fibers, such as eyelashes, including a step for applying on said keratin materials, in particular the eyelashes, at least a cosmetic composition as defined hereinabove.

The invention also relates to a non-therapeutic cosmetic method for makeup for keratin materials, in particular keratin fibers, such as eyelashes, including a step for applying on the keratin materials, in particular on the eyelashes, a cosmetic composition as defined hereinabove.

The present invention also relates to a method for coating keratin fibers, and in particular makeup for eyelashes, comprising a step of applying a cosmetic composition for coating keratin fibers as described hereinabove.

The present invention also relates to the use of a composition as defined hereinabove, for obtaining a deposit on the eyelashes of satisfactory black intensity.

Packaging and Application Assembly or Kit

The present invention also relates to an assembly, or kit, for packaging and application of a cosmetic composition for coating keratin fibers comprising:
a device for packaging said cosmetic composition for coating keratin fibers such as described hereinabove,
an applicator of said composition.

Said applicator can be integral with a gripping member forming a cover for said packaging device. In other words, said applicator can be mounted in a removable position on said device between a sealed position and a released position of an opening for the distribution of the device for packaging said composition

An assembly for coating keratin fibers adapted to the invention can comprise in particular an applicator configured to apply said cosmetic composition for coating keratin fibers, and where applicable a device for packaging adapted to receive said composition.

Applicator

The applicator comprises means making it possible to smooth and/or separate the keratin fibers, such as the eyelashes or the eyebrows, in particular in the form of teeth, bristles, pins or other reliefs.

The applicator is arranged to apply the composition on the eyelashes or the eyebrows, and can comprise for example a brush or a comb.

The applicator can further be used for the finishing of the makeup, on a region of the eyelashes or of the eyebrows with makeup or loaded with the composition.

The brush can comprise a twisted core and bristles taken between the spires of the core, or be carried out yet in another manner.

The comb is for example made of a single piece by molding of plastic material.

In certain embodiments, the element for application is mounted at the end of a rod, which can be flexible, which can contribute to improving the comfort during application.

Packaging Device

The packaging device comprises a receptacle intended to house the composition for coating keratin fibers. This composition can then be taken in the receptacle by immersing the application into the latter.

This applicator can be integral with an element for closing the receptacle. This closing element can form a member for gripping the applicator. This gripping member can form a cover to be mounted removably on said receptacle by any suitable means such as by screwing, snap-fitting, press-fitting or other. Such a receptacle can therefore house in a reversible manner said applicator.

This receptacle may be provided with a squeezing device suited to remove the excess product taken by the applicator.

A method for applying the composition according to the invention on the eyelashes or on the eyebrows can also comprise the following steps:
forming a deposit of the cosmetic composition on the eyelashes or the eyebrows,
leaving the deposit on the eyelashes or on the eyebrows, with the deposit able to dry.

Note that according to another embodiment, the applicator may form a product receptacle. In such a case, a receptacle may for example be provided in the gripping member and an internal channel can connect on the inside this gripping member to the application elements in relief.

Finally, note that the assembly for packaging and applying can have the form of a kit, with the applicator and the packaging device able to be housed separately under the same packaging article.

Throughout the application, the term “comprising a” or “including a” means “comprising at least one” or “including at least one”, unless otherwise specified.

Throughout the above description, unless specified otherwise, the term “between x and y” refers to an inclusive range, i.e. the values x and y are included in the range.

### EXAMPLES

A composition according to the invention with a pasty fat (C1) as defined above and a comparative composition without this pasty fat (example 2), as well as a comparative composition without this pasty fat but with another kind of pasty fat (example 3) were prepared and tested for the black intensity thereof.

Preparation Protocol for Said Compositions

In a tank, at ambient temperature, all the ingredients below were introduced, with the exception of the acrylic film-forming polymer (DAITOSOL 5000 AD), in a volume of water corresponding to 20% of the total water.

The mixture obtained was heated to 95° C. and stirred for 20 minutes. The rest of the water was then added and the mixture was homogenized and emulsified for 15 minutes with mechanical stirring (blades+turbine).

The mixture was then allowed to cool with stirring up to 40° C. The acrylic film-forming polymer (DAITOSOL 5000 AD) was then added.

The final mixture was homogenized with the blades and allowed to cool to 20° C.

Cosmetic compositions with and without pasty fat (C1) according to the invention were prepared according to the tables hereinbelow.

Example 1 according to the invention comprises a pasty fat (C1) as described above, while the comparative example 2 does not comprise a pasty fat (C1) according to the invention, and the comparative example 3 comprises a pasty fat other than the pasty fat (C1) according to the invention.

| | | Trade | Example 1 | Example 2 |
|---|---|---|---|---|
| Ingredients | Supplier | name | (Invention) | (comparative) |
| Disodium EDTA | BASF | EDETA BD | 0.2 | 0.2 |
| Iron oxides | SUN | SUNPURO BLACK | 7.14 | 7.14 |
| | | IRON OXIDE C33- | | |
| | | 7001 | | |
| Sodium dehydroacetate | SHANGHAI | SODIUM | 0.3 | 0.3 |
| | XINWANG | DEHYDROACETATE | | |
| | POLYMER | | | |
| | MATERIAL | | | |
| Phenoxyethanol | SEPPIC | SEPICIDE LD | 0.84 | 0.84 |
| Carnauba wax | STRAHL & | CARNAUBA WAX #1 | 3.5 | 3.5 |
| (COPERNICIA CERIFERA WAX) | PITSCH | FLAKES N.F. SP 63 | | |
| BEESWAX | KOSTER | WHITE BEESWAX | 4.4 | 4.4 |
| | KEUNEN | (GR B 889) | | |
| Glyceryl behenate (and) tribehenin | GATTEFOSSE | Compritol 888 CG | 2 | 0 |
| (and) glyceryl behenate | | ATO | | |
| Cetyl alcohol | BASF | LANETTE 16 | 2 | 2 |
| Paraffin | BAERLOCHER | AFFINE 56-58 | 11.12 | 11.12 |
| | | PASTILLES | | |
| Hydrogenated jojoba oil | DESERT | JOJOBA WAX | 0.25 | 0.25 |
| | WHALE | FLAKES | | |
| Hydrogenated palm oil | SIO (ADM) | GV 60 | 0.25 | 0.25 |
| Hydroxyethylcellulose | AMERCHOL | CELLOSIZE QP | 0.75 | 0.75 |
| | (DOW | 4400 H | | |
| | CHEMICAL) | | | |
| Acacia gum (ACACIA SENEGAL | NEXIRA | SPRAYGUM SC10 | 0.63 | 0.63 |
| GUM) | | | | |
| Ethylene/acrylic acid copolymer | HONEYWELL | ASENSA SC 401 | 2.78 | 2.78 |
| Acrylate copolymer in aqueous | DAITO KASEI | DAITOSOL 5000 AD | 10 | 10 |
| dispersion at 50% | KOGYO | | | |
| Ethylenediamine/stearyl dimer | CRODA | OLEOCRAFT LP-10- | 0.5 | 0.5 |
| dilinoleate copolymer | | PA-(MV) | | |
| Water | | | 45.94 | 47.94 |
| Caprylyl glycol | DR | DERMOSOFT | 0.3 | 0.3 |
| | STRAETMANS | OCTIOL | | |
| Steareth-2 | EVONIK | TEGO ALKANOL S2 | 2.1 | 2.1 |
| | GOLDSCHMIDT | | | |
| Potassium cetylphosphate | DSM | AMPHISOL K | 5 | 5 |
| | NUTRITIONAL | (0452130) | | |
| | PRODUCTS | | | |

| | | Trade | Example 3 |
|---|---|---|---|
| Ingredients | Supplier | name | (comparative) |
| Disodium EDTA | BASF | EDETA BD | 0.2 |
| Iron oxides | SUN | SUNPURO BLACK | 7.14 |
| | | IRON OXIDE C33- | |
| | | 7001 | |
| Sodium dehydroacetate | SHANGHAI | SODIUM | 0.3 |
| | XINWANG | DEHYDROACETATE | |
| | POLYMER | | |
| | MATERIAL | | |
| Phenoxyethanol | SEPPIC | SEPICIDE LD | 0.84 |
| Carnauba wax | STRAHL & | CARNAUBA WAX #1 | 3.5 |
| (COPERNICIA CERIFERA WAX) | PITSCH | FLAKES N.F. SP 63 | |
| BEESWAX | KOSTER | WHITE BEESWAX | 4.4 |
| | KEUNEN | (GR B 889) | |
| Glyceryl behenate (and) tribehenin | GATTEFOSSE | Compritol 888 CG | 0 |
| (and) glyceryl behenate | | ATO | |
| TRIBEHENIN PEG-20 ESTERS | GATTEFOSSE | EMULIUM 22 | 2 |
| Cetyl alcohol | BASF | LANETTE 16 | 2 |
| Paraffin | BAERLOCHER | AFFINE 56-58 | 11.12 |
| | | PASTILLES | |
| Hydrogenated jojoba oil | DESERT | JOJOBA WAX | 0.25 |
| | WHALE | FLAKES | |
| Hydrogenated palm oil | SIO (ADM) | GV 60 | 0.25 |
| Hydroxyethylcellulose | AMERCHOL | CELLOSIZE QP | 0.75 |
| | (DOW | 4400 H | |
| | CHEMICAL) | | |
| Acacia gum (ACACIA SENEGAL | NEXIRA | SPRAYGUM SC10 | 0.63 |
| GUM) | | | |
| Ethylene/acrylic acid copolymer | HONEYWELL | ASENSA SC 401 | 2.78 |
| Acrylate copolymer in aqueous | DAITO KASEI | DAITOSOL 5000 AD | 10 |
| dispersion at 50% | KOGYO | | |
| Ethylenediamine/stearyl dimer | CRODA | OLEOCRAFT LP-10- | 0.5 |
| dilinoleate copolymer | | PA-(MV) | |
| Water | | | 45.94 |
| Caprylyl glycol | DR | DERMOSOFT | 0.3 |
| | STRAETMANS | OCTIOL | |
| Steareth-2 | EVONIK | TEGO ALKANOL S2 | 2.1 |
| | GOLDSCHMIDT | | |
| Potassium cetylphosphate | DSM | AMPHISOL K | 5 |
| | NUTRITIONAL | (0452130) | |
| | PRODUCTS | | |

It is understood that, within the scope of the present invention, the weight percentages given, for a compound or a family of compounds, are always expressed by weight of dry matter of the compound in question (unless specified otherwise).

Protocols and Results Relating to Black Intensity

The mascara composition in example 1 according to the invention and the mascara composition in examples 2 and 3 outside the invention were tested according to a black intensity measurement protocol.

Black Measurement Protocol

The apparatus used to measure the black value is Spectro-guide Sphere Gloss/BYK. The apparatus was set to measure the color without the gloss (Gloss OFF and D65/10°).

A film is applied on a glass slide with a 150 μm deposit thickness. The mascara deposit is allowed to dry for 24 hours at ambient temperature. Finally, measurement is made with the colorimeter on a dry film with the plastic support of the colorimeter.

The results obtained are as follows:

| | Example 1 | Example 2 | Example 3 |
|---|---|---|---|
| | (invention) | (comparative) | (comparative) |
| L | 22.23 | 22.97 | 23.43 |
| a | 0.24 | 0.21 | 0.13 |
| b | −0.76 | −0.82 | −1.08 |

The blackness of the composition comprising the compound C1 (example 1), namely Glyceryl behenate (and) tribehenin (and) glyceryl behenate (Compritol 888 CG ATO (GATTEFOSSE)), is much higher (lower “L” value) than example 2 which does not contain it.

The blackness of the composition comprising the compound C1 (example 1) is also much higher (lower “L” value) than example 3 which does not contain it but contains another kind of pasty fat (Emulium 22).

The difference in blackness is also visually perceptible.

Indeed, it can be seen that the composition of example 1 (according to the invention) has a higher blackness in mass in a recipient and also between two glass plates, in comparison with the composition of example 3 (with Emulium 22).

1. A composition in the form of a wax-in-water emulsion, comprising at least one pasty fat chosen from (poly)esters of at least one C₂₀-C₁₀₀ fatty acid and at least one polyol said pasty fat being a mixture comprising glyceryl monobehenate, glyceryl di-behenate and glyceryl tri-behenate,
wherein the wax content is strictly greater than 5% by weight with respect to the total weight of said composition.

2. The composition according to claim 1, wherein the pasty fat content is greater than or equal to 0.1 by weight with respect to the total weight of said composition.

3. The composition according to claim 1, further comprising polybutene.

4. The composition according to claim 3, wherein the polybutene content is between 0.1% and 10% by weight with respect to the total weight of said composition.

5. The composition according to claim 1, comprising at least one film-forming polymer.

6. The composition according to claim 1, further comprising at least one additional pasty fat chosen from glycerol oligomer esters.

7. The composition according to claim 1, wherein the water content is greater than or equal to 40% by weight with respect to the total weight of said composition.

8. The composition according to claim 1, in which the wax content is between 5.5% and 20% by weight with respect to the total weight of said composition.

9. The composition according to claim 1, comprising at least one lipophilic gelling agent.

10. The composition according to claim 1, comprising at least one emulsifying system comprising at least one surfactant, preferably chosen from non-ionic surfactants, anionic surfactants, and mixtures thereof, notably with a total content ranging from 4% to 14% by weight with respect to the total weight of said composition.

11. The composition according to claim 10, wherein the surfactant(s) is/are chosen from non-ionic surfactants from anionic surfactants and from mixtures thereof.

12. A method for coating keratin fibers comprising a step of applying a composition according to claim 1.

13. The composition according to claim 1, wherein the (poly)ester is behenic acid and the at least one polyol is glycerol.

14. The composition according to claim 1, wherein the pasty fat content is from 0.5% to 5% by weight with respect to the total weight of said composition.

15. The composition according to claim 1, wherein the pasty fat content is from 1% to 3% by weight with respect to the total weight of said composition.

16. The composition according to claim 2, further comprising polybutene.

17. The composition according to claim 2, comprising at least one film-forming polymer.

18. The composition according to claim 3, comprising at least one film-forming polymer.

19. The composition according to claim 4, comprising at least one film-forming polymer.

20. The composition according to claim 2, further comprising at least one additional pasty fat chosen from glycerol oligomer esters.
